(19) United States
(12) Patent Application Publication
(10) Pub. No.: US 2022/0192606 A1
(43) Pub. Date: Jun. 23, 2022

(54) Systems and Methods for Acquiring and Analyzing High-Speed Eye Movement Data

(71) Applicant: Eye Tech Digital Systems, Inc., Mesa, AZ (US)

(72) Inventor: Robert C. Chappell, Mesa, AZ (US)

(21) Appl. No.: 17/560,631

(22) Filed: Dec. 23, 2021

Related U.S. Application Data
(60) Provisional application No. 63/129,859, filed on Dec. 23, 2020.

(51) Int. Cl. A61B 5/00; A61B 5/11

(52) U.S. Cl. CPC A61B 5/7275; A61B 5/7267; A61B 5/4088; A61B 5/742; A61B 5/0077; A61B 5/1114; A61B 5/1126; A61B 5/4082

(57) ABSTRACT

An eye-movement data acquisition system includes an illumination source configured to produce infrared light and a camera assembly configured to receive a portion of the infrared light reflected from a user's face during activation of the infrared illumination source. The camera assembly includes a rolling shutter sensor configured to produce individual scan line images associated with the user's eyes at a line sampling rate. A processor is communicatively coupled to the camera assembly and the illumination sources and is configured to produce eye-movement data based on the individual scan line images.

### CROSS-REFERENCE TO RELATED APPLICATIONS

This application claims priority to U.S. Provisional Patent Application No. 63/129,859, filed Dec. 23, 2020, the entire contents of which are hereby incorporated by reference.

### TECHNICAL FIELD

The present invention relates, generally, to eye-tracking systems and methods and, more particularly, to the acquisition and analysis of high-speed eye movement data using image sensors.

### BACKGROUND

The behavior of an individual's eyes can be linked to cognitive processes, such as attention, memory, and decision-making. Accordingly, changes in eye movements over time may accompany and help predict the changes that occur in the brain due to aging and neurodegeneration. Such changes may thus be early leading indicators of Alzheimer's disease, Parkinson's disease, and the like.

Eye-tracking systems—such as those used in conjunction with desktop computers, laptops, tablets, virtual reality headsets, and other computing devices that include a display—generally include one or more illuminators configured to direct infrared light to the user's eyes and an image sensor that captures the images for further processing. By determining the relative locations of the user's pupils and the corneal reflections produced by the illuminators, the eye-tracking system can accurately predict the user's gaze point on the display.

While it would be advantageous to use such eye-tracking systems to collect eye tracking data and images of a user's face for medical purposes, it is difficult or impossible to do so because the data acquisition speed of typical eye-tracking systems are not fast enough to capture a wide range of anomalies. That is, the eye tracking sampling rate of most systems is limited by the framerate of the sensor and the speed of the associated data transfer circuits and processing.

During conventional eye tracking, an entire frame is captured, downloaded, and processed to give one sample point for eye position. The framerate of the sensor can be increased by decreasing the frame size, especially the number of lines read out from the sensor. However, the framerate is ultimately limited by the need to capture enough of the eye for tracking and head movement and by limitations of the sensor hardware. Thus, the sample rate is typically limited to several hundred Hertz. For certain neurological conditions, sampling rates in this range are not sufficient.

Accordingly, there is a long-felt need for systems and methods for high-speed/low-noise processing and analysis of eye-movement data in the context of medical diagnoses. Systems and methods are therefore needed that overcome these and other limitations of the prior art.

### SUMMARY OF THE INVENTION

Various embodiments of the present invention relate to systems and methods for, inter alia, sampling a user's eye movement at the line rate of the camera, thereby providing an estimate of the eye position on every line read from the camera (rather than every frame). In this way, sample rates in the tens of thousands of hertz can be achieved.

In some embodiments, by capturing and processing one line of pixels across the pupil, the system can estimate the center of the pupil on each line along an axis defined by the orientation of the sensor. For many neurological tests, this sample rate is sufficient for capturing movement, at least in that dimension. A variety of image sensors, such as one or more rolling-shutter sensors, may be used to implement the illustrated embodiments.

In some embodiments, when it is desirable to capture movement along another axis (e.g., 90° relative to the first axis), then a second camera with its sensor rotated 90 degrees relative to the first camera could also be used to scan the eye at the same time. That is, one camera provides the x-position and the other camera provides they-position, and these positions are correlated based on time stamps to derive the (x, y) position over time. In further embodiments, a secondary, conventional-speed “finding camera” is used to assist the primary camera in determining the location of the eye.

### BRIEF DESCRIPTION OF THE DRAWING FIGURES

The present invention will hereinafter be described in conjunction with the appended drawing figures, wherein like numerals denote like elements, and:

FIG. 1 is a conceptual diagram illustrating line-by-line sampling of any eye in accordance with various embodiments of the present invention;

FIGS. 2A and 2B illustrate the use of two cameras oriented at a 90-degree angle relative to each other in accordance with various embodiments;

FIG. 3 is a conceptual block diagram illustrating an eye-tracking system in accordance with various embodiments; and

FIGS. 4A and 4B illustrate the use of an eye-tracking system in accordance with various embodiments.

### DETAILED DESCRIPTION OF PREFERRED

### Exemplary Embodiments

The present subject matter generally relates to improved systems and methods for high-speed acquisition of eye-movement data for the purposes of diagnosing medical conditions. In that regard, the following detailed description is merely exemplary in nature and is not intended to limit the inventions or the application and uses of the inventions described herein. Furthermore, there is no intention to be bound by any theory presented in the preceding background or the following detailed description. In the interest of brevity, conventional techniques and components related to eye-tracking algorithms, image sensors, machine learning systems, cognitive diseases, and digital image processing may not be described in detail herein.

As mentioned briefly above, embodiments of the present invention relate to systems and methods for, inter alia, sampling a user's eye movement at the line rate of the camera (e.g., on the order of tens of thousands of Hz) to thereby providing an estimate of the eye position on every line read from the camera.

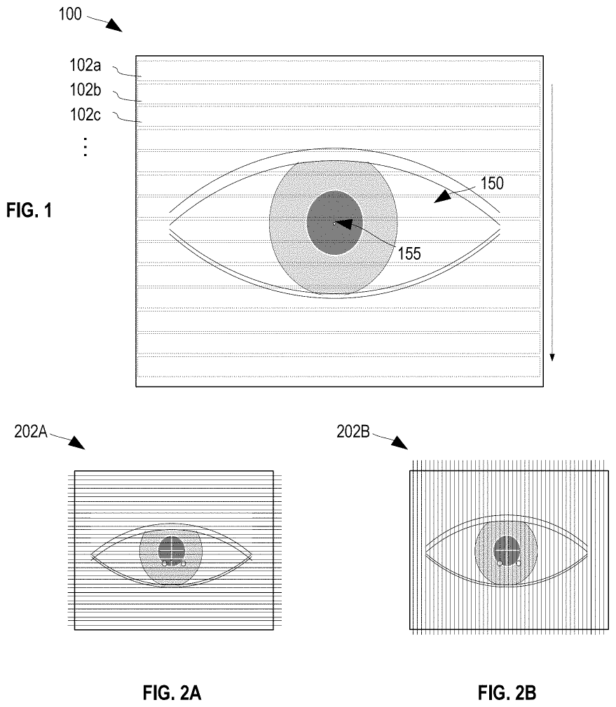

More particularly, FIG. 1 illustrates an image 100 of a user's eye 150 as it might appear when viewed head-on by an image sensor—i.e., when the user is looking straight ahead at the camera lens. Also illustrated in FIG. 1 are individual scan lines (e.g., 102a, 102b, 102c), corresponding to the top-to-bottom scanning pattern of a typical sensor. That is, horizontal line 102a is acquired first, horizontal line 102b is acquired second, and so on. As used herein, the phrase “rolling shutter sensor” refers to any sensor (e.g., a CMOS sensor) that does not necessarily expose the entire sensor for capture at one time (i.e., a “global shutter,” as in typical CCD sensors), but rather exposes different parts of the sensor (e.g., a single line) at different points in time.

When viewed head-on as in FIG. 1, the pupil 155 appears as a nearly perfect circle. By capturing and processing one line of pixels across the pupil, the system can estimate the center of the pupil on each line. That is, the left and right edges of pupil 155 can be determined from this single scan, and the average of those two values can be used as an estimate of the center (or epicenter) of the pupil along the horizontal axis. When the user's eye makes even a small, fast movement, the difference in centers observed by the system between line scans can be captured and analyzed. More particularly, if the sampling period is known, and the change in center values are known, then the rate of the user's eye during that sample can be estimated using conventional mathematical methods. The system may then be trained to recognize certain neurological conditions through supervised learning—i.e., by observing the patterns of eye movements in individuals exhibiting known medical conditions.

Because each line is sampled at a different time, there will generally be a slight positional change or apparent distortion of the circular pupil shape (particularly in rolling shutter systems) due to large scale movement of the user. However, because of the high sampling rate, this large scale movement can be separated from the microsaccades and other small scale movements of the pupil 155.

In some embodiments, when it is also desirable to capture movement along another axis (e.g., 90° relative to the first dimension) then two (or more) cameras may be employed. This is illustrated in FIGS. 2A and 2B, in which two cameras oriented at 90 degrees relative to each other are used to acquire horizontal line data (202A) and vertical line data (202B) simultaneously. Using time-stamps for scans 202A and 202B, the x and y coordinates at any given time can be derived, and this information can be used to observed eye movement over time.

If the user is not staring directly at the camera, but is instead looking off at some angle, then the pupil will appear as an ellipse, which will appear in the line-by-line position data as a slope. However, this slope will be repeated from frame-to-frame, and thus can be accounted for mathematically. In addition, there may be structural patterns in the user's iris that causes the pupil edge to be geometrically anomalous. These anomalies will also show up as repeating patterns from frame-to-frame and can be removed either by subtraction in the time domain or filtering at the frequency of the framerate. The scan information that remains after such filtering corresponds to non-repeating patterns that are unrelated to framerate, are the movements of the eye that are important for medical diagnostics.

When acquiring images in this way, is has been observed that there will often be periodic holes in the data. That is, for each frame, there will be some time when the scanning lines are outside the pupil or the sensor is internally scanning to catch up on its timing at the end of a frame. This can be accounted for in the data analysis itself, and as long as the patterns the system need to see are regularly captured and analyzed, then these gaps or missing data are not material to the analysis. Furthermore, these gaps can be minimized by configuring the scanned region such that the pupil fills as much of the camera image as possible. In some embodiments, this is accomplished by using a longer focal length lens and moving the user closer, and/or reducing the frame size setting on the sensor. This can be taken to a limit wherein they-dimension of the frame size is actually less than the pupil height. In such a case, every line read from the sensor would provide position data, but there would still be some gaps in the data at the end of a frame due to the blanking time required by the sensor.

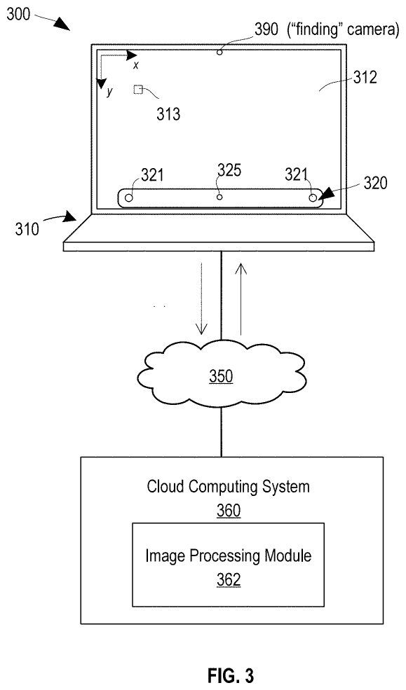

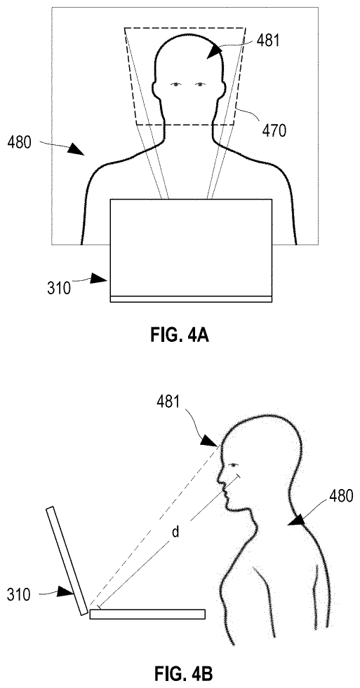

In accordance with one embodiment, two (or more) cameras are used, where one camera has a wider field-of-view (a “finding camera”) and a longer focal length. While the present invention may be implemented in a variety of ways, FIG. 3 in conjunction with FIGS. 4A and 4B illustrates just one example of a system 300, which will now be described.

As shown in FIG. 3, system 300 includes some form of computing device 310 (e.g., a desktop computer, tablet computer, laptop, smart-phone, head-mounted display, television panels, dashboard-mounted automotive systems, or the like) having an eye-tracking assembly 320 coupled to, integrated into, or otherwise associated with device 310. System 300 also includes a “finding” camera 390, which may be located in any convenient location (not limited to the top center as shown). The eye-tracking assembly 320 is configured to observe the facial region 481 (FIG. 4A) of a user (alternatively referred to as a “patient” or “experimental subject”) within a field of view 470 and, through the techniques described above, track the location and movement of the user's gaze (or “gaze point”) 313 on a display (or “screen”) 312 of computing device 310. The gaze point 313 may be characterized, for example, by a tuple (x, y) specifying linear coordinates (in pixels, centimeters, or other suitable unit) relative to an arbitrary reference point on display screen 312 (e.g., the upper left corner, as shown). As also described above, high speed movement of the user's pupil(s) may also be sampled, in addition to the gaze itself.

In the illustrated embodiment, eye-tracking assembly 320 includes one or more infrared (IR) light emitting diodes (LEDs) 321 positioned to illuminate facial region 481 of user 480. Assembly 320 further includes one or more cameras 325 configured to acquire, at a suitable frame-rate, digital images corresponding to region 481 of the user's face. As described above, camera 325 might be a rolling shutter camera or other image sensor device capable of providing line-by-line data of the user's eyes.

In some embodiments, the image data may be processed locally (i.e., within computing device 310) using an installed software client. In some embodiments, however, eye motion sampling is accomplished using an image processing module or modules 362 that are remote from computing device 310—e.g., hosted within a cloud computing system 360 communicatively coupled to computing device 310 over a network 350 (e.g., the Internet). In such embodiments, image processing module 362 performs the computationally complex operations necessary to determine the gaze point and is then transmitted back (as eye and gaze data) over the network to computing device 310. An example cloud-based eye-tracking system that may employed in the context of the present invention may be found, for example, in U.S. patent application Ser. No. 16/434,830, entitled “Devices and Methods for Reducing Computational and Transmission Latencies in Cloud Based Eye Tracking Systems,” filed Jun. 7, 2019, the contents of which are hereby incorporated by reference.

In contrast to traditional eye-tracking, in which the gaze data is processed in near real-time to determine the gaze point, in the context of analyzing microsaccades it is not necessary to process the data immediately. That is, the high-speed data may be acquired and stored during testing, and then later processed—either locally or via a cloud computing platform—to investigate possible neurodegeneration or other conditions correlated to the observed eye movements.

In accordance with one embodiment, a moving region-of-interest may be used to adjust the censor region of interest from frame-to-frame so that it covers just the pupil area and minimizes gaps in the data. This configuration can be used for the x-dimension data and one more camera could be added to do the same thing for y-dimension data. One camera would give the frame-by-frame eye position in x and y dimensions and the other two cameras would give the line by line position with one of them rotated 90 degrees with respect to the other.

In accordance with an alternate embodiment, another approach for achieving moderately high framerates is to use two cameras that both produce data at the frame level. One of the cameras has a wider field of view and gives the eye position frame-to-frame. The other camera is set with the smallest possible frame size that still encompasses the entire pupil and runs as fast as possible for that small frame size. This results in data with no gaps at hundreds of hertz to possibly greater than 1000 hertz. While such an embodiment is not as fast as collecting data on every line as described above, it could potentially give higher quality data. The sensor with the smallest region-of-interest would use a moving region-of-interest that is positioned based on information from the other camera or cameras.

Eye movements may be categorized as pursuit eye movements, saccadic eye movements, and vergence eye movements, as is known in the art. In accordance with the present invention, one or more of these types of movements may be used as a correlative to a medical condition, such as various neurological disorders (Alzheimer's disease, ataxia, Huntington's disease, Parkinson's disease, motor neuron disease, multiple system atrophy, progressive supranuclear palsy, and any other disorder that manifests to some extent in a distinctive eye movement pattern).

The systems, modules, and other components described herein may employ one or more machine learning or predictive analytics models to assist in predicting and/or diagnosing medical conditions. In this regard, the phrase “machine learning” model is used without loss of generality to refer to any result of an analysis that is designed to make some form of prediction, such as predicting the state of a response variable, clustering patients, determining association rules, and performing anomaly detection. Thus, for example, the term “machine learning” refers to models that undergo supervised, unsupervised, semi-supervised, and/or reinforcement learning. Such models may perform classification (e.g., binary or multiclass classification), regression, clustering, dimensionality reduction, and/or such tasks. Examples of such models include, without limitation, artificial neural networks (ANN) (such as a recurrent neural networks (RNN) and convolutional neural network (CNN)), decision tree models (such as classification and regression trees (CART)), ensemble learning models (such as boosting, bootstrapped aggregation, gradient boosting machines, and random forests), Bayesian network models (e.g., naive Bayes), principal component analysis (PCA), support vector machines (SVM), clustering models (such as K-nearest-neighbor, K-means, expectation maximization, hierarchical clustering, etc.), linear discriminant analysis models.

In summary, what have been described are systems and methods for high-speed acquisition of eye-movement data for the purposes of diagnosing medical conditions.

In accordance with one embodiment, an eye-movement data acquisition system includes: an illumination source configured to produce infrared light; a camera assembly configured to receive a portion of the infrared light reflected from a user's face during activation of the infrared illumination source, wherein the camera assembly includes a rolling shutter sensor configured to produce individual scan line images associated with the user's eyes at a line sampling rate; and a processor communicatively coupled to the camera assembly and the illumination sources, the processor configured to produce eye-movement data based on the individual scan line images.

In one embodiment, the processor is further configured to produce an output indicative of a likelihood of the user having a medical condition based on the eye-movement data. In one embodiment, the output is produced by a previously-trained machine learning model.

In one embodiment, the medical condition is a neurodegenerative disease selected from the group consisting of Alzheimer's disease, ataxia, Huntington's disease, Parkinson's disease, motor neuron disease, multiple system atrophy, and progressive supranuclear palsy.

In one embodiment, the line sampling rate is greater than 10000 Hz. In some embodiments, the processor is further configured to determine the center of a user's pupil within each scan line images. In some embodiments, the system includes a second camera assembly configured to produce scan line images that are perpendicular to the scan line images produced by the first camera assembly. In other embodiments, a third non-rolling-shutter camera is configured to assist the first camera assembly in determining the location of the user's eyes.

A method of diagnosing a medical condition in a user in accordance with one embodiment includes: providing a first infrared illumination source; receiving, with a camera assembly configured, a portion of the infrared light reflected from a user's face during activation of the infrared illumination source, wherein the camera assembly includes a rolling shutter sensor; producing, with the rolling shutter sensor, individual scan line images associated with the user's eyes at a line sampling rate; producing, with a processor, eye-movement data based on the individual scan line images; and producing an output indicative of a likelihood of the user having a medical condition based on the eye-movement data.

In one embodiment, the output is produced by a previously-trained machine learning model. In another embodiment, the medical condition is a neurodegenerative disease such as Alzheimer's disease, ataxia, Huntington's disease, Parkinson's disease, motor neuron disease, multiple system atrophy, and progressive supranuclear palsy. In some embodiments, the line sampling rate is greater than 10000 Hz.

A medical diagnosis system in accordance with one embodiment includes: a display; an illumination source configured to produce infrared light; a camera assembly configured to receive a portion of the infrared light reflected from a user's face during activation of the infrared illumination source, wherein the camera assembly includes a rolling shutter sensor configured to produce individual scan line images associated with the user's eyes at a line sampling rate greater than 10000 Hz; and a processor communicatively coupled to the camera assembly and the illumination sources, the processor configured to produce eye-movement data based on the individual scan line images and to produce an output indicative of a likelihood of the user having a medical condition based on the eye-movement data.

As used herein, the terms “module” or “controller” refer to any hardware, software, firmware, electronic control component, processing logic, and/or processor device, individually or in any combination, including without limitation: application specific integrated circuits (ASICs), field-programmable gate-arrays (FPGAs), dedicated neural network devices (e.g., Google Tensor Processing Units), electronic circuits, processors (shared, dedicated, or group) configured to execute one or more software or firmware programs, a combinational logic circuit, and/or other suitable components that provide the described functionality.

As used herein, the word “exemplary” means “serving as an example, instance, or illustration.” Any implementation described herein as “exemplary” is not necessarily to be construed as preferred or advantageous over other implementations, nor is it intended to be construed as a model that must be literally duplicated.

While the foregoing detailed description will provide those skilled in the art with a convenient road map for implementing various embodiments of the invention, it should be appreciated that the particular embodiments described above are only examples, and are not intended to limit the scope, applicability, or configuration of the invention in any way. To the contrary, various changes may be made in the function and arrangement of elements described without departing from the scope of the invention.

1. An eye-movement data acquisition system comprising:
an illumination source configured to produce infrared light;
a camera assembly configured to receive a portion of the infrared light reflected from a user's face during activation of the infrared illumination source, wherein the camera assembly includes a rolling shutter sensor configured to produce individual scan line images associated with the user's eyes at a line sampling rate; and
a processor communicatively coupled to the camera assembly and the illumination sources, the processor configured to produce eye-movement data based on the individual scan line images.

2. The system of claim 1, wherein the processor is further configured to produce an output indicative of a likelihood of the user having a medical condition based on the eye-movement data.

3. The system of claim 2, wherein the output is produced by a previously-trained machine learning model.

4. The system of claim 3, wherein the medical condition is a neurodegenerative disease.

5. The system of claim 4, wherein the neurodegenerative disease is selected from the group consisting of Alzheimer's disease, ataxia, Huntington's disease, Parkinson's disease, motor neuron disease, multiple system atrophy, and progressive supranuclear palsy.

6. The system of claim 1, wherein the line sampling rate is greater than 10000 Hz.

7. The system of claim 1, wherein the processor is further configured to determine the center of a user's pupil within each scan line images.

8. The system of claim 1, further including a second camera assembly configured to produce scan line images that are perpendicular to the scan line images produced by the first camera assembly.

9. The system of claim 1, further including a third non-rolling-shutter camera configured to assist the first camera assembly in determining the location of the user's eyes.

10. A method of diagnosing a medical condition in a user, the method comprising:
providing a first infrared illumination source;
receiving, with a camera assembly configured, a portion of the infrared light reflected from a user's face during activation of the infrared illumination source, wherein the camera assembly includes a rolling shutter sensor;
producing, with the rolling shutter sensor, individual scan line images associated with the user's eyes at a line sampling rate;
producing, with a processor, eye-movement data based on the individual scan line images; and
producing an output indicative of a likelihood of the user having a medical condition based on the eye-movement data.

11. The method of claim 10, wherein the output is produced by a previously-trained machine learning model.

12. The method of claim 10, wherein the medical condition is a neurodegenerative disease.

13. The method of claim 12, wherein the neurodegenerative disease is selected from the group consisting of Alzheimer's disease, ataxia, Huntington's disease, Parkinson's disease, motor neuron disease, multiple system atrophy, and progressive supranuclear palsy.

14. The method of claim 10, wherein the line sampling rate is greater than 10000 Hz.

15. The method of claim 10, further including determining the center of a user's pupil within each scan line images.

16. The method of claim 10, further including producing scan line images, with a second camera assembly, that are perpendicular to the scan line images produced by the first camera assembly.

17. The system of claim 1, further determining the location of the user's eyes with a third, non-rolling-shutter camera assembly.

18. A medical diagnosis system comprising:
a display;
an illumination source configured to produce infrared light;
a camera assembly configured to receive a portion of the infrared light reflected from a user's face during activation of the infrared illumination source, wherein the camera assembly includes a rolling shutter sensor configured to produce individual scan line images associated with the user's eyes at a line sampling rate greater than 10000 Hz; and
a processor communicatively coupled to the camera assembly and the illumination sources, the processor configured to produce eye-movement data based on the individual scan line images and to produce an output indicative of a likelihood of the user having a medical condition based on the eye-movement data.

19. The system of claim 18, wherein the output is produced by a previously-trained machine learning model, and the medical condition is a neurodegenerative disease.

20. The system of claim 18, further including a second camera assembly configured to produce scan line images that are perpendicular to the scan line images produced by the first camera assembly.
